(19) United States
(12) Patent Application Publication
(10) Pub. No.: US 2025/0263664 A1
(43) Pub. Date: Aug. 21, 2025

(54) Method for Culturing Stem Cells Using Mitochondria-Activating Ingredient NRF and Mixed solution for Culturing Stem Cells

(71) Applicant: Yu-Lin Yang, Kaohsiung City (TW)

(72) Inventors: Yu-Lin Yang, Kaohsiung City (TW); Yao-Hsien Wang, Kaohsiung City (TW); Chia-Hua Yang, Kaohsiung City (TW)

(21) Appl. No.: 18/786,815

(22) Filed: Jul. 29, 2024

(30) Foreign Application Priority Data
Feb. 16, 2024 (TW) 113105508

(51) Int. Cl. C12N 5/0775; C12N 5/00

(52) U.S. Cl. CPC C12N 5/0662; C12N 5/0018; C12N 2500/02; C12N 2500/76; C12N 2523/00

(57) ABSTRACT

A method for culturing stem cells is used to solve the problem of loss of stem cell functionality after passaging. The method includes forming a mixed solution by adding an extract of Salvia miltiorrhiza into a basal medium. The stem cells are then cultured in the mixed solution. By the use of the extract of Salvia miltiorrhiza, division and differentiation capacities of the stem cells can be maintained even after passaging. The mixed solution for culturing the stem cells is also disclosed.

### CROSS REFERENCE TO RELATED APPLICATIONS

The application claims the benefit of Taiwan application serial No. 113105508, filed on Feb. 16, 2024, the subject matter of which are incorporated herein by reference.

### BACKGROUND OF THE INVENTION

### 1. Field of the Invention

The present invention generally relates to a method for culturing cells and, more particularly, to a method for culturing stem cells. The present invention also relates to a mixed solution for culturing the stem cells.

### 2. Description of the Related Art

Stem cells refer to primitive and undifferentiated cells that exist in multicellular organisms. Stem cells can differentiate into various types of cells and proliferate to produce more of the same stem cells. Adult stem cells are undifferentiated cells that spread throughout the body after development. They can replace dying cells and regenerate damaged tissues through cell division. Adult stem cells exist in specific tissues of the adult body, such as bone marrow mesenchymal stem cell, hematopoietic stem cell, neural stem cell, etc.

Mesenchymal stem cells (MSCs) are adult stem cells with the potential to differentiate into various tissues. They are known to be isolated from umbilical cord blood, adipose tissue and bone marrow, and can differentiate into various cell types such as osteoblasts, chondroblasts, adipocytes and neuroectodermal cells. In addition, MSCs are believed to have healing capabilities and can be used in regenerative medicine therapies.

As present, the most common source of MSCs is adult bone marrow [known as bone marrow mesenchymal stem cells (BM-MSCs)]. However, the number of adult-derived MSCs is rare, and being subcultured for multiple times are needed to obtain enough MSCs for clinical application. Moreover, the process of in vitro subculture will cause aging of adult cells and affect the division and differentiation capabilities of MSCs. In light of this, a mixed solution for culturing stem cells and a method for culturing stem cells utilizing the mixed solution are still needed.

### SUMMARY OF THE INVENTION

It is therefore an objective of the present invention to provide a method for culturing stem cells, which is used to obtain stem cells with normal division and differentiation capacities.

It is another objective of the present invention to provide a mixed solution for culturing stem cells, which is applied to the aforementioned method.

As used herein, the term “a”, “an” or “one” for describing the number of the elements and members of the present invention is used for convenience, providing the general meaning of the scope of the present invention, and should be interpreted to include one or at least one. Furthermore, unless explicitly indicated otherwise, the concept of a single component also includes the case of plural components.

One embodiment of the present invention discloses a method for culturing stem cells. The method comprises: adding an extract of Salvia miltiorrhiza to a basal medium to form a mixed solution; and culturing the stem cells in the mixed solution. For example, the stem cells can be cultured in an environment with a carbon dioxide (CO₂) concentration of from 5% to 10%, a temperature of from 35° C. to 39° C., and a relative humidity of from 90% to 99%. Moreover, the extract of Salvia miltiorrhiza can be extracted from a root sample of Salvia miltiorrhiza by water at a temperature of from 70° C. to 100° C.

Accordingly, in the method for culturing stem cells according to the present invention, by the use of the extract of Salvia miltiorrhiza, the mitochondrial activity of the stem cells is upregulated, the energy production of the stem cells is regulated, and thus the stem cells will have enough energy to divide and differentiate. As such, aging of the stem cells can be prevented, and the rejuvenation ability of the stem cells can be promoted. Therefore, the stem cells can maintain normal division and differentiation capacities.

In the method for culturing the stem cells, the mixed solution can comprise the extract of Salvia miltiorrhiza in a concentration of from 1 g/L to 100 g/L. As such, by the appropriate amount of the extract of Salvia miltiorrhiza, the cell vitality of the stem cells can be enhanced, and aging of the stem cells can be prevented.

In the method for culturing the stem cells, the stem cells can be mesenchymal stem cells (MSCs). As such, the MSCs with good rejuvenation ability can be obtained, and the MSCs can be used for cell therapy.

Another embodiment of the present invention discloses a mixed solution for culturing stem cells. The mixed solution comprises: a basal medium; and an extract of Salvia miltiorrhiza. For example, the extract of Salvia miltiorrhiza can be extracted from a root sample of Salvia miltiorrhiza by water at a temperature of from 70° C. to 100° C.

Accordingly, by the extract of Salvia miltiorrhiza, the mitochondrial activity of the stem cells is upregulated, the energy production of the stem cells is regulated, and thus the stem cells will have enough energy to divide and differentiate. As such, aging of the stem cells can be prevented, and the rejuvenation ability of the stem cells can be promoted. Therefore, the stem cells can maintain normal division and differentiation capacities.

In the mixed solution for culturing the stem cells, the mixed solution can comprise the extract of Salvia miltiorrhiza in a concentration of from 1 g/L to 100 g/L. As such, by the appropriate amount of the extract of Salvia miltiorrhiza, the cell vitality of the stem cells can be enhanced, and aging of the stem cells can be prevented.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present invention will become more fully understood from the detailed description given hereinafter and the accompanying drawings which are given by way of illustration only, and thus are not limitative of the present invention, and wherein:

FIG. 1 depicts a flow chart illustrating a method for culturing stem cells according to an embodiment of present invention.

FIG. 2 depicts a line chart illustrating the adenosine triphosphate (ATP) content in mesenchymal stem cells (MSCs) cultured in mixed solutions containing an extract of Salvia miltiorrhiza (mitochondria-activating ingredient NRF) in different concentration in trial (A).

FIG. 3 depicts a bar chart illustrating the cell number of MSCs of groups B0-B1 on the day of seeding and the cell number of MSCs after 3 days of culturing in trial (B).

FIG. 4 depicts a microscopic image of adipocytes differentiated from the MSCs of group C0 in trial (C).

FIG. 5 depicts a microscopic image of adipocytes differentiated from the MSCs of group C1 in trial (C).

FIG. 6 depicts a microscopic image of chondroblasts differentiated from the MSCs of group C0 in trial (C).

FIG. 7 depicts a microscopic image of chondroblasts differentiated from the MSCs of group C1 in trial (C).

FIG. 8 depicts a microscopic image of osteocytes differentiated from the MSCs of group C0 in trial (C).

FIG. 9 depicts a microscopic image of osteocytes differentiated from the MSCs of group C1 in trial (C).

### DETAILED DESCRIPTION OF THE INVENTION

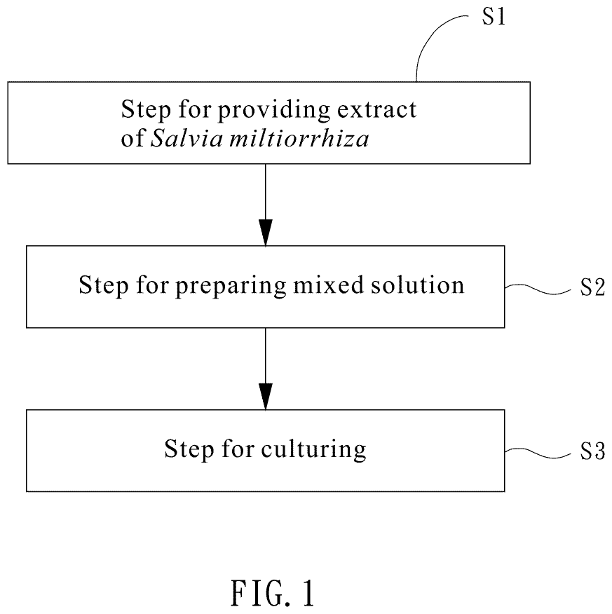

Referring to FIG. 1, a method for culturing stem cells according to an embodiment of the present invention can comprise a step for providing an extract of Salvia miltiorrhiza S1, a step for preparing a mixed solution S2 and a step for culturing S3.

Specifically, in the step for providing the extract of Salvia miltiorrhiza S1, the extract of Salvia miltiorrhiza is provided. By the extract of Salvia miltiorrhiza, aging of the stem cells can be prevented, and the rejuvenation ability of the stem cells can be promoted. Therefore, the stem cells can maintain normal division and differentiation capacities. In the following, the extract of Salvia miltiorrhiza will be referred as a mitochondria-activating ingredient NRF.

The mitochondria-activating ingredient NRF can be preferably obtained by a method with the following steps: providing a sample of Salvia miltiorrhiza; extracting the sample of Salvia miltiorrhiza by water as an extractant; and concentrating a rough extract of Salvia miltiorrhiza to obtain the mitochondria-activating ingredient NRF.

Preferably, a root sample of Salvia miltiorrhiza can be used as the sample of Salvia miltiorrhiza. Moreover, before being extracted by water, the sample of Salvia miltiorrhiza can be dried to form a dried sample of Salvia miltiorrhiza with a moisture content being less than or equal to 5%.

For example, for per 50 g of the sample of Salvia miltiorrhiza, the extractant of 1,000 mL can be used to mix with the sample of Salvia miltiorrhiza, forming a mixture. Then, in order to completely dissolving the active gradients of the sample of Salvia miltiorrhiza in the extractant, the sample of Salvia miltiorrhiza is extracted at a temperature of from about 70° C. to about 100° C. for a total extraction time of 60 minutes, forming the raw extract of Salvia miltiorrhiza, which can be appreciated by a person having ordinary skill in the art. Detail description is omitted to avoid redundancy.

After being cooled and filtrated, the raw extract of Salvia miltiorrhiza can be freeze dried to form the mitochondria-activating ingredient NRF with water content less than 5%. By the procedure, the active ingredients of the mitochondria-activating ingredient NRF can be concentrated, and by administration of a few amount of the mitochondria-activating ingredient NRF can achieve the effect. In this embodiment, through the aforementioned process, approximately 200 g of the mitochondria-activating ingredient NRF (dry weight) can be obtained from the 1 kg of the sample of Salvia miltiorrhiza.

Moreover, the mitochondria-activating ingredient NRF can be mixed with an extract of Medicago sativa. The extract of Medicago sativa can activate mitochondria together with the extract of Salvia miltiorrhiza.

In the step for preparing the mixed solution S2, a basal medium is first prepared. The basal medium can be any commercial medium known to be used for culturing stem cells. For example, the basal medium can comprise amino acids such as glycine (Gly), valine (Val), leucine (Leu), isoleucine (Ile), phenylalanine (Phe), tryptophan (Trp), tyrosine (Tyr), histidine (His), lysine (Lys), methionine (Met), arginine (Arg), serine (Ser), threonine (Thr) and/or cysteine (Cys). The basal medium can also comprise vitamins such as thiamine (vitamin B1), riboflavin (vitamin B2), niacin (vitamin B3), pantothenic acid (vitamin B5), pyridoxine (vitamin B6), inositol (vitamin B8), folic acid (vitamin B9) and/or choline chloride. The basal medium can further comprise inorganic salts such as sodium bicarbonate (NaHCO₃), magnesium sulfate (MgSO₄), monosodium phosphate (NaH₂PO₄), sodium chloride (NaCl), potassium chloride (KCl), calcium chloride (CaCl₂)) and/or ferric nitrate (Fe(NO₃)₃). In this embodiment, the basal medium is LG-DMEM medium (purchased from Gibco; CAT NO: 11054001), which further comprises glucose and sodium pyruvate, with 10% of fetal bovine serum (FBS). The formula of the LG-DMEM medium without FBS is shown in TABLE 1.

TABLE 1

| | Concentration |
|---|---|
| Ingredients | (mg/L) |
| Glycine | 30.0 |
| L-Arginine hydrochloride | 84.0 |
| L-Cystine 2HCl | 63.0 |
| L-Histidine hydrochloride-H₂O | 42.0 |
| L-Isoleucine | 105.0 |
| L-Leucine | 105.0 |
| L-Lysine hydrochloride | 146.0 |
| L-Methionine | 30.0 |
| L-Phenylalanine | 66.0 |
| L-Serine | 42.0 |
| L-Threonine | 95.0 |
| L-Tryptophan | 16.0 |
| L-Tyrosine disodium salt dihydrate | 104.0 |
| L-Valine | 94.0 |
| Choline chloride | 4.0 |
| D-Calcium pantothenate | 4.0 |
| Folic acid | 4.0 |
| Niacinamide | 4.0 |
| Pyridoxine hydrochloride | 4.0 |
| Riboflavin | 0.4 |
| Thiamine hydrochloride | 4.0 |
| i-Inositol | 7.2 |
| Calcium chloride (CaCl₂ (anhydrous)) | 200.0 |
| Ferric nitrate (Fe(NO₃)₃•9H₂O) | 0.1 |
| Magnesium sulfate (MgSO₄ (anhydrous)) | 97.67 |
| Potassium chloride (KCl) | 400.0 |
| Sodium bicarbonate (NaHCO₃) | 3700.0 |
| Sodium chloride (NaCl) | 6400.0 |
| Sodium phosphate monobasic (NaH₂PO₄•H₂O) | 125.0 |
| D-glucose | 1000.0 |
| Sodium pyruvate | 110.0 |
| Water | Up to 1 L |

Next, the mitochondria-activating ingredient NRF can be added to the basal medium to form the mixed solution. For example, the mixed solution can comprise the mitochondria-activating ingredient NRF in a concentration of from 1 g/L to 100 g/L, and the mixed solution can preferably comprise the mitochondria-activating ingredient NRF in a concentration of 1 g/L.

In the step for culturing S3, the stem cells can be cultured in the mixed solution. For example, the stem cells can be mesenchymal stem cells (MSCs), such as MSCs from bone marrow, adipose tissue, umbilical cord, synovium, skeletal muscle, placenta and peripheral blood.

Specifically, an appropriate amount of freshly prepared mixed solution is added to a culture vessel (e.g., a culture dish, a culture flask or a culture plate), and the stem cells are seeded to the mixed solution in a concentration of 4×10⁷ cells/L. The mixed solution is then placed in an incubator with a temperature of from 35° C. to 39° C., a relative humidity of 90% to 99%, and a carbon dioxide (CO₂) concentration of from 5% to 10%, ensuring a pH value of the mixed solution can be from 7.2 to 7.4.

During the process of culturing the stem cells, freshly prepared mixed solution can be replaced about every 2 days. When the stem cells in the culture vessel reach about 70-80% confluency, the stem cells can be subcultured.

In the method for culturing stem cells according to the embodiment, by the mitochondria-activating ingredient NRF, even after being subcultured for multiple times, the stem cells still have good activity, and the rejuvenation ability of the stem cells can be promoted. Therefore, the stem cells can maintain normal division and differentiation capacities.

It is worthy to note that when culturing the stem cells in the mixed solution containing the mitochondria-activating ingredient NRF, it is not necessary to start from the time that the stem cells are thawed. A worker can first subculture the stem cells for several times using the basal medium, and then culture the stem cells using the mixed solution containing the mitochondria-activating ingredient NRF at a certain generation, which can be adjusted by a person having ordinary skill in the art.

To demonstrate that the addition of the mitochondria-activating ingredient NRF can upregulate the mitochondrial activity of the stem cells, the following trials are carried out.

### Trial (A).

In trial (A), the LG-DMEM medium with FBS is used as the basal medium, and the mitochondria-activating ingredient NRF is added to the basal medium in a concentration of 0.1%, 0.5% and 1.0% (w/v), respectively, to form the mixed solution. The MSCs are cultured in the mixed solution for 24 hours. After collecting the MSCs, the adenosine triphosphate (ATP) content in mesenchymal stem cells (MSCs) is detected by luminescent ATP detection assay kit (purchased from Abcam; CAT NO: ab113849).

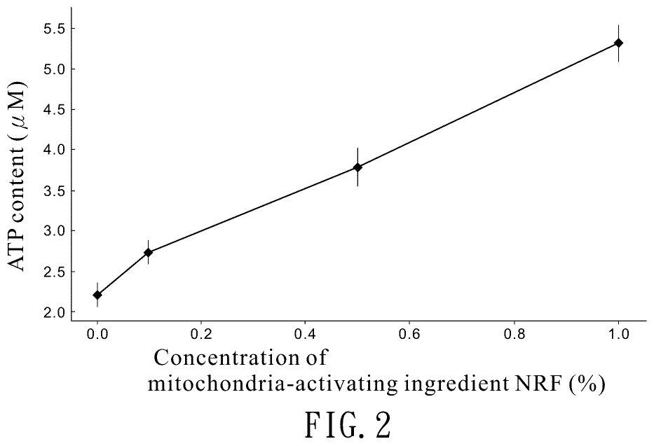

Referring to FIG. 2, the addition of the mitochondria-activating ingredient NRF can improve the ATP content in MSCs, and the improvement of the ATP content is positive correlation with the amount of the mitochondria-activating ingredient NRF (p<0.05).

Moreover, to demonstrate the addition of the mitochondria-activating ingredient NRF can improve the activity of the stem cells, the MSCs are cultured in the basal medium (the LG-DMEM medium with FBS). After subculturing for 9 time (i.e., the MSCs with a passage number of 10), the mixed solution with the basal medium and the mitochondria-activating ingredient NRF (0.1% (w/v)), instead of the basal medium, is used to culture the MSCs. The following trials are carried out.

### Trial (B)

In trial (B), the MSCs cultured in the basal medium are used as group B0, while the MSCs cultured in the mixed solution containing the mitochondria-activating ingredient NRF (0.1% (w/v)) are used as group B1. 1.5×10⁵ cells are seeded, and the MSCs are collected after 3 days of culturing. The cell number of the MSCs of groups B0-B1 are calculated.

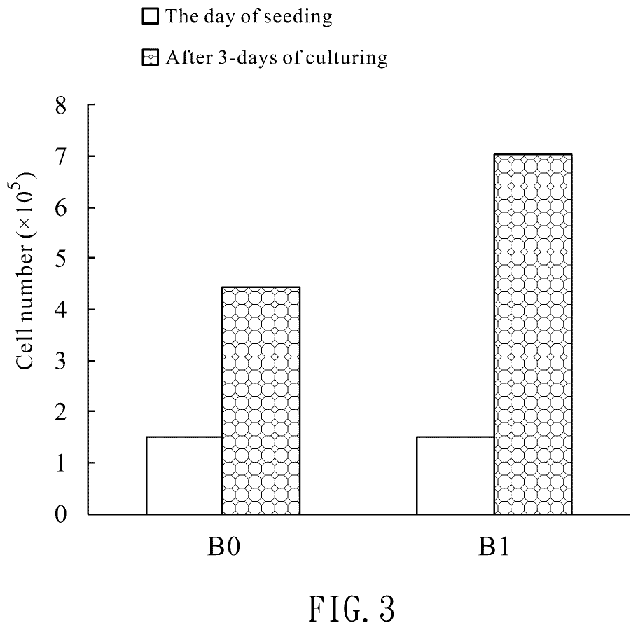

Referring to FIG. 3, after 3 days, the cell number of the MSCs cultured in the basal medium is about 4.5×10⁵, while the cell number of the MSCs cultured in the mixed solution is about 7×10⁵, indicating that the addition of the mitochondria-activating ingredient NRF (0.1% (w/v)) can help improve the division capability of the MSCs that have been subcultured for multiple times.

### Trial (C)

In trial (C), the MSCs cultured in the basal medium are used as group C0, while the MSCs culture in the mixed solution containing the mitochondria-activating ingredient NRF (0.1% (w/v)) are used as group C1. The MSCs are then induced to differentiate into adipocyte, chondroblast and osteocyte, respectively.

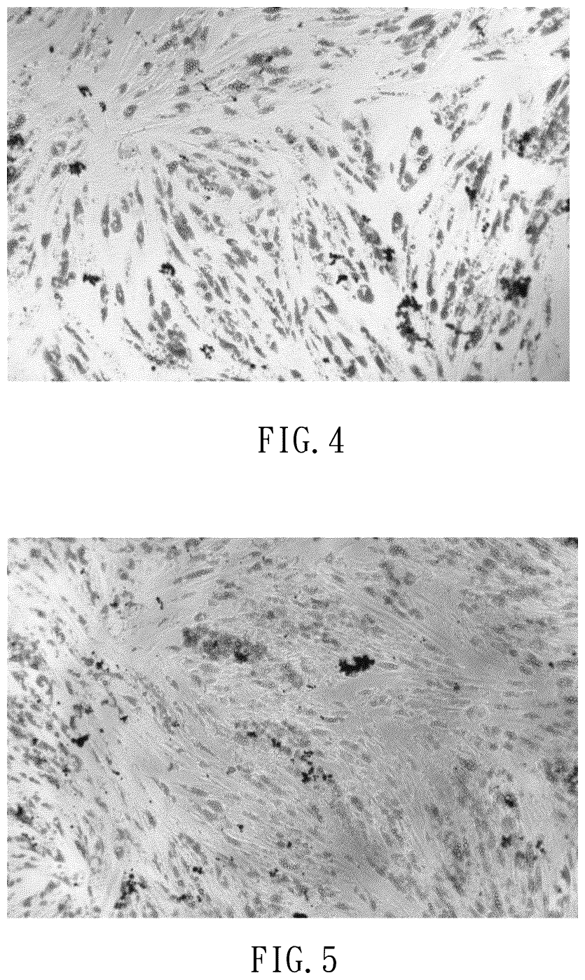

Referring to FIGS. 4-5, the results of Oil Red O staining show that both the MSCs of groups C0-C1 can successfully differentiate into adipocytes, among which the cell number of adipocytes differentiated by MSCs of group C1 is larger, indicating that the addition of the mitochondria-activating ingredient NRF can help improve the differentiation capability into adipocytes of the MSCs that have been subcultured for multiple times.

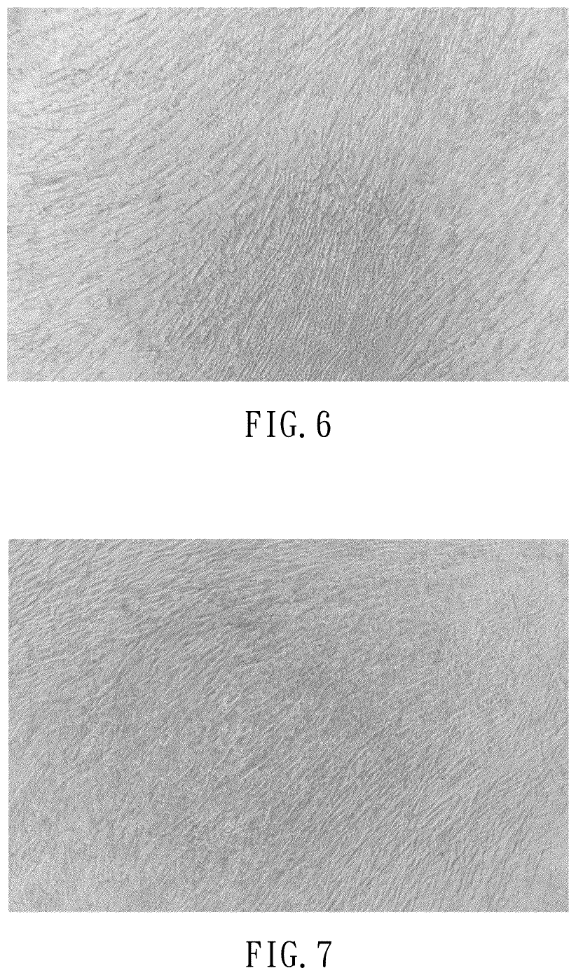

Referring to FIGS. 6-7, the results of Alcian blue staining show that both the MSCs of groups C0-C1 can successfully differentiate into chondroblasts, among which the cell number of chondroblasts differentiated by MSCs of group C1 is larger, indicating that the addition of the mitochondria-activating ingredient NRF can help improve the differentiation capability into chondroblasts of the MSCs that have been subcultured for multiple times.

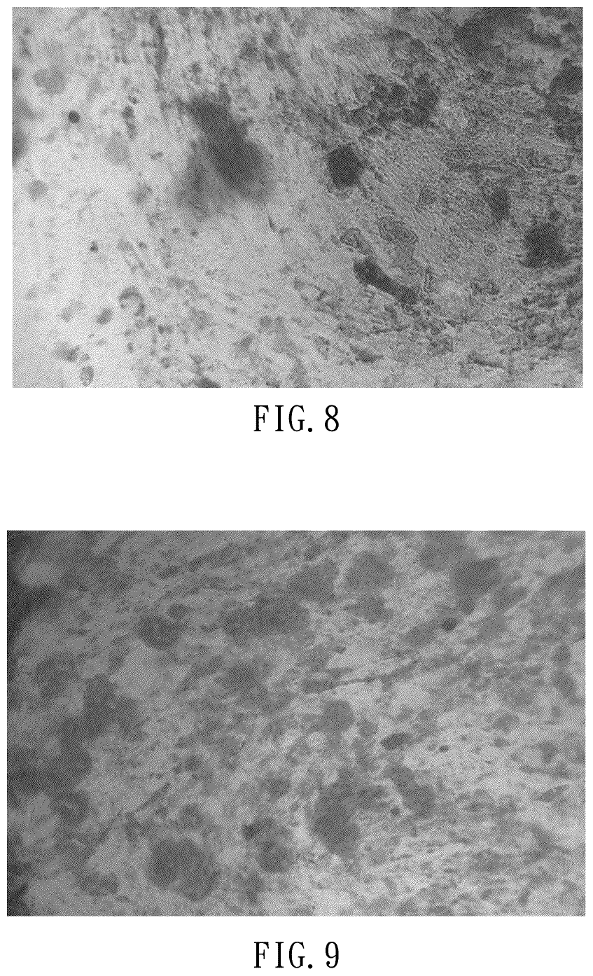

Referring to FIGS. 8-9, the results of Alizarin Red S staining show that both the MSCs of groups C0-C1 can successfully differentiate into osteocytes, among which the cell number of osteocytes differentiated by MSCs of group C1 is larger, indicating that the addition of the mitochondria-activating ingredient NRF can help improve the differentiation capability into osteocytes of the MSCs that have been subcultured for multiple times.

Accordingly, by the use of the extract of Salvia miltiorrhiza, the mitochondrial activity of the stem cells is upregulated, the energy production of the stem cells is regulated, and thus the stem cells will have enough energy to divide and differentiate. As such, aging of the stem cells can be prevented, and the rejuvenation ability of the stem cells can be promoted. Therefore, the stem cells can maintain normal division and differentiation capacities.

Although the invention has been described in detail with reference to its presently preferable embodiment, it will be understood by one of ordinary skill in the art that various modifications can be made without departing from the spirit and the scope of the invention, as set forth in the appended claims.

What is claimed is:

1. A method for culturing stem cells, comprising:
adding an extract of Salvia miltiorrhiza to a basal medium to form a mixed solution; and
culturing the stem cells by the mixed solution.

2. The method for culturing the stem cells as claimed in claim 1, wherein the extract of Salvia miltiorrhiza is extracted from a root sample of Salvia miltiorrhiza by water at a temperature of from 70° C. to 100° C.

3. The method for culturing the stem cells as claimed in claim 1, wherein the mixed solution comprises the extract of Salvia miltiorrhiza in a concentration of from 1 g/L to 100 g/L.

4. The method for culturing the stem cells as claimed in claim 2, wherein the mixed solution comprises the extract of Salvia miltiorrhiza in a concentration of from 1 g/L to 100 g/L.

5. The method for culturing the stem cells as claimed in claim 1, wherein the stem cells are cultured in an environment with a carbon dioxide (CO₂) concentration of from 5% to 10%, a temperature of from 35° C. to 39° C., and a relative humidity of from 90% to 99%.

6. The method for culturing the stem cells as claimed in claim 2, wherein the stem cells are cultured in an environment with a carbon dioxide (CO₂) concentration of from 5% to 10%, a temperature of from 35° C. to 39° C., and a relative humidity of from 90% to 99%.

7. The method for culturing the stem cells as claimed in claim 3, wherein the stem cells are cultured in an environment with a carbon dioxide (CO₂) concentration of from 5% to 10%, a temperature of from 35° C. to 39° C., and a relative humidity of from 90% to 99%.

8. The method for culturing the stem cells as claimed in claim 1, wherein the stem cells are mesenchymal stem cells (MSCs).

9. A mixed solution for culturing stem cells, comprising:
a basal medium; and
an extract of Salvia miltiorrhiza.

10. The mixed solution for culturing the stem cells as claimed in claim 9,
wherein the extract of Salvia miltiorrhiza is extracted from a root sample of Salvia miltiorrhiza by water at a temperature of from 70° C. to 100° C.

11. The mixed solution for culturing the stem cells as claimed in claim 9, wherein the mixed solution comprises the extract of Salvia miltiorrhiza in a concentration of from 1 g/L to 100 g/L.

12. The mixed solution for culturing the stem cells as claimed in claim 10, wherein the mixed solution comprises the extract of Salvia miltiorrhiza in a concentration of from 1 g/L to 100 g/L.
